(19) United States
(12) Patent Application Publication
(10) Pub. No.: US 2011/0137208 A1
(43) Pub. Date: Jun. 9, 2011

(54) DEVICE AND METHOD FOR AUTOMATICALLY SAMPLING AND MEASURING BLOOD ANALYTES

(75) Inventor: Timothy Walter Valk, Boynton Beach, FL (US)

(73) Assignee: ADMETSYS CORPORATION, Boynton Beach, FL (US)

(21) Appl. No.: 13/055,207

(22) PCT Filed: Jul. 23, 2009
(86) PCT No.: PCT/US2009/051486

§ 371 Date: Jan. 21, 2011

Related U.S. Application Data
(60) Provisional application No. 61/083,450, filed on Jul. 24, 2008.

(51) Int. Cl. A61B 5/151

(52) U.S. Cl. 600/583

(57) ABSTRACT

The present invention includes a device and method used for automated sampling and measurement of blood analytes, such as blood glucose, from a subject patient. The blood sampling and measurement may be performed automatically by the device on a patient without requiring user intervention or supervision. The sampling and measuring device comprises a sensor unit operably attached to a replaceable cartridge. The replaceable cartridge provides a disposable body that contains a number of lancets and test strips used for collecting and analyzing a blood sample. The sensor unit contains an actuator and microcontroller that fires the lancet and collects blood sample data from the test strips. The blood sampling and measurement process may be initiated by an external controller or may be autonomously initiated in a specific interval. A further embodiment involves integration and use of the sampling and measurement device within an automated monitoring and treatment system.

### FIELD OF THE INVENTION

The present invention relates to a device and method for automatically sampling and measuring blood analytes, such as glucose, in a patient.

### BACKGROUND OF THE INVENTION

Hospital bound patients must have measurements of various physiologic analytes measured and tested on a routine and sometimes frequent basis. The vast majority of these measurements are done manually by hospital nurses and other staff thereby creating constraints on hospital staff time and an overall burden on the health care system. While analytes, such as triglycerides, total cholesterol, HDL-cholesterol, fibrinogen, hemoglobin, ferritin, glucose, and the like may be required to be measured during a patient's hospital stay, the present disclosure uses blood glucose sampling and measurement as an exemplary embodiment of the invention.

Hyperglycemia is a frequent consequence of severe illness, occurring in both diabetic and non-diabetic patients, due to altered metabolic and hormonal systems, impaired gastrointestinal motility, altered cardiac function, increased catecholamine production, altered hepatic gluconeogenesis, relative insulin resistance, and increased corticosteroid levels. Symptoms associated with elevated levels of blood glucose include dehydration, weakness, an increased risk of infection and poor healing, frequent urination, and thirst. Infusion of insulin has proven an effective method for treating hyperglycemia. However, insulin infusion without proper glucose level monitoring can lead to problems with hypoglycemia.

Hypoglycemia in both diabetic and non-diabetic patients is one physiological condition that is monitored in an intensive care and/or other acute medical setting. Hypoglycemia is a common problem with severely ill patients and is defined as the fall of blood and tissue glucose levels to below 72 mg/dL. Symptoms associated with decreased levels of blood and tissue glucose levels include weakness, sweating, loss of concentration, shakiness, nervousness, change in vision, loss of consciousness, possible seizures, and neurological sequelae such as paralysis and death. Treatment in the case of both hyperglycemia and hypoglycemia involves monitoring and controlling the patient's glucose level.

Data provided in medical studies indicates that hypoglycemia occurs in 3.8%-4% of all patients when glucose is measured every 2 hours. In other words, the average patient has a hypoglycemic episode every 2 to 4 days. The mean time that patients spent in the intensive care unit in these studies was between 2.5 and 10 days. Thus, theoretically, the average patient would have at least 1 and possibly up to 5 episodes of hypoglycemia during their intensive care unit stay. To reduce the risk of hypoglycemia, the burden is on healthcare staff to monitor patient glucose levels every 1 to 1.5 hours. In addition, healthcare staff must implement increasingly complex procedures to monitor and control patients' glucose levels. This level of attention by healthcare professionals is not practical for busy hospital intensive care units. Furthermore, as a result of increases in medical malpractice claims, hospitals are reluctant to treat hyperglycemia vigorously, fearing that any hypoglycemia might be attributed to such treatment.

Measurements of glucose from blood continue to be the most accurate and reliable to monitor the aforementioned conditions. The current widely used blood measurement technique (as well as for other blood analytes such as total and HDL-cholesterol) is the manual finger-prick. This method is simple, safe, and reliable. However, while sufficient for home monitoring use, in a hospital environment the burden on staff is enormous. The tedious and time-consuming nature of repeated testing limits the practical frequency of glucose measurements in hospital care. For instance, the manual finger-prick method may involve periodic measurements (typically hourly) of the patient's blood glucose level. The nursing staff must then obtain orders from a doctor to adjust the amount of insulin being delivered to the patient in an effort to maintain the patient's blood glucose level within a desired range. This method is time consuming, costly, and prone to error.

Current blood glucose sampling methods use indwelling venous and arterial catheters. Such approaches introduce the possibility of additional medical complications such as clotting, infection, and immune response. This is especially true when used over longer periods or in seriously ill individuals.

Automation of the widely-used current finger prick technique, without the need for manual intervention, would mitigate hospital staff constraints without introducing new medical complexity. Therefore, there exists a need for an automated glucose system that utilizes technology known to be safe and reliable, but that relieves the burden of manual intervention associated with the individual monitoring of glucose and other analyte levels in a patient.

### BRIEF SUMMARY OF THE INVENTION

The presently disclosed sampling and measuring device in accordance with the present invention uses electromechanical automation to sample and measure blood glucose and other analytes of a patient. The presently disclosed method for sampling and measuring blood analytes uses this sampling and measuring device to obtain repeated automated measurements over a period of time.

It is an object of the invention to provide a device and method for sampling and measuring blood analytes in a patient over an extended period of time without the need for manual intervention. This sampling and measuring process may be initiated by an external automated controller, by another sampling and measuring device, or by self-contained processing logic within the device.

It is a further object of the invention to enable hospital staff to situate a small sampling and sensing apparatus on a patient, adjust its sizing to fit the patient properly, affix a replaceable supply cartridge to the apparatus, and leave the apparatus to automatically monitor blood glucose levels and other blood analytes, as needed, for an extended period of time without manual intervention.

It is a further object of the invention to provide an automated device for sampling and measuring blood analytes, including a sensor unit structured to be positioned on a patient body, the sensor unit having electronic circuitry, lancet firing means, and variable pressure control means; and a replaceable cartridge having a plurality of consumable products disposed therein, the consumable products including one or more lancets and one or more test strips for measuring a blood analyte of the patient; wherein the replaceable cartridge and the sensor unit are structured to temporarily mate with one another via an attachment means such that the replaceable cartridge is removable from the sensor unit; wherein the electronic circuitry enables automated blood extraction and analysis of a blood analyte from the patient body iteratively over time without need for manual intervention; and wherein the automated blood extraction and analysis is performed through electronically controlled use of the variable pressure control means, the lancet firing means, the one or more lancets, and the one or more blood test strips.

It is a further object of the present invention to provide an automated device for sampling and measuring a blood analyte of a patient, having a sensor unit structured to be positioned adjacent a measurement site of a patient, the sensor unit including an upper portion and a lower portion operably connected thereto, the sensor unit including an lancet firing means; and a replaceable cartridge in mating relationship with the sensor unit via an attachment means such that the replaceable cartridge is removable from the sensor unit, the replaceable cartridge housing a plurality of consumable products disposed therein for producing a blood sample, the consumable products including one or more lancets and one or more test strips for measuring the blood analyte of the patient; a microcontroller and electronic circuitry operably coupled to the sensor unit and capable of controlling use of the lancets and test strips relative to the measurement site; a set of electronic instructions executable by the microcontroller such that upon execution, the electronic instructions causes the microcontroller to initiate a sequence including selecting a lancet for deployment at a measurement site, firing the lancet to obtain a blood sample from the measurement site, and collecting a blood sample from the measurement site onto a test strip; wherein the microcontroller receives inputs from the test strip to determine the blood analyte and further wherein the electronic instructions cause the microcontroller to initiate the sequence without the need for manual intervention.

It is a further object of the present invention to provide an automated system for monitoring blood analytes of a patient, including a sampling and measurement device structured to be positioned adjacent a measurement site of a patient, the device housing a replaceable supply of consumable products including a plurality of lancets and a plurality of test strips for the measurement of blood analytes; a microcontroller operably coupled to the sampling and measurement device and capable of controlling the plurality of lancets and plurality of test strips relative to the measurement site; a set of electronic instructions executable by the microcontroller such that upon execution, the electronic instructions causes the microcontroller to initiate a sequence including selecting a lancet and test strip for use at the measurement site, firing the lancet to obtain a blood sample from the measurement site, collecting a blood sample from the measurement site; and depositing the blood sample onto the test strip; wherein the microcontroller processes a electrochemical reaction from the test strip to determine the level of blood analytes and further wherein the electronic instructions cause the microcontroller to initiate the sequence without the need for manual intervention.

It is a further object of the present invention to provide a method for deploying an automated device for sampling and measuring blood analytes from a patient, including: positioning an automated sampling and measuring device proximate to a measurement site on a patient, the sampling and measuring device configured to obtain blood analyte measurements from blood samples initiated with an automated process; providing a set of replaceable materials to the automated sampling and measuring device, the set of replaceable materials including a plurality of reactive areas, each reactive area including one or more lancets and one or more test strips; performing an automated blood analyte sampling and measurement using a blood sample obtained from the measurement site, the blood sample introduced to one of the plurality of the reactive areas provided to the automated sampling and measuring device; and automatically repeating the step of performing a blood analyte measurement using a unused reactive area from the plurality of reactive areas, thereby performing a new blood analyte sampling and measurement at the measurement site without user intervention.

It is a further object of the present invention to provide a method for sampling and measuring of blood analytes from a patient with an automated device, including: affixing an automated sampling and measuring device to a patient, the sampling and measuring device accessing a supply of consumable products including a plurality of lancets and a plurality of test strips; executing a set of electronic instructions by a microcontroller within the sampling and measuring device, the execution of the electronic instructions causing the microcontroller to initiate a sequence for sampling and measuring a level of a blood analyte with the sampling and measuring device, the sequence including: applying pressure proximate to a measurement site on the patient; firing a lancet to obtain a blood sample from the measurement site; exposing a test strip to the blood sample from the measurement site; and obtaining an electrochemical measurement of the blood analyte level from the test strip; wherein the set of electronic instructions for initiating the sampling and measuring the blood analyte level are executed by the microcontroller iteratively over a period of time or upon request, thereby enabling the sampling and measuring device to perform a series of automated sampling and measuring events without need for manual intervention.

With use of the various embodiments of the present invention, blood analyte measurements may be recorded, displayed, or sent directly to a therapeutic control device to adjust infusion or other treatment. Further embodiments of the present invention include control of the sampling and measuring apparatus and appropriate treatment through use of an external monitoring and treatment system.

### BRIEF DESCRIPTION OF THE DRAWINGS

FIG. 1A is a diagram illustrating a closed-loop treatment cycle facilitated by operation of one embodiment of the present invention;

FIG. 1B is a diagram illustrating the monitored portion of the closed-loop treatment cycle facilitated by operation of one embodiment of the present invention;

FIG. 2A is an illustration of a sampling and measuring device adapted to attach to a finger of a testing subject according to one embodiment of the present invention;

FIG. 2B is an illustration of a sampling and measuring device adapted to attach to multiple fingers of a testing subject according to one embodiment of the present invention;

FIG. 2C is an illustration of a sampling and measuring device adapted to attach to the palm of a testing subject according to one embodiment of the present invention;

FIG. 3 is a perspective view of a sampling and measuring device having a sensor unit and a replaceable cartridge in accordance with one embodiment of the present invention;

FIG. 4 is an exploded view of the sensor unit and the replaceable cartridge within a sampling and measuring device in accordance with one embodiment of the present invention;

FIG. 5A depicts a cross section of two adjacent reactive test areas within the sampling and measuring device used to obtain blood analytes in accordance with one embodiment of the present invention;

FIGS. 5B-5C depict another view of reactive test areas within the sampling and measuring device used to obtain blood analytes in accordance with one embodiment of the present invention;

FIGS. 6A-6D illustrate one exemplary technique of obtaining blood analyte measurements by applying pressure, firing a lancet, retracting the lancet, measuring blood chemistry, and releasing pressure in accordance with one embodiment of the present invention;

FIG. 6E illustrates an alternative technique of applying pressure to a measurement site with a single compress in accordance with one embodiment of the present invention;

FIG. 7 illustrates multiple blood analyte sampling and measuring devices linked together in accordance with one embodiment of the present invention;

FIG. 8 is a block diagram illustrating the electronic components within the sensor unit and the removable cartridge of the sampling and measuring device in accordance with one embodiment of the present invention;

FIG. 9 is a high-level circuit diagram depicting a circuit used for functional control of the sampling and measuring device in accordance with one embodiment of the present invention;

FIG. 10 is a high-level circuit diagram depicting components and subcircuits within the sampling and measuring device in accordance with one embodiment of the present invention;

FIG. 11 is a flowchart illustrating a method for deploying an automated device for sampling and measuring blood analytes from a patient in accordance with one embodiment of the present invention;

FIG. 12 is a flowchart illustrating a method for sampling and measuring blood analytes from a patient with an automated device in accordance with one embodiment of the present invention;

FIG. 13 is a block diagram illustrating exemplary components of an external controller that may be used in accordance with one embodiment of the present invention;

FIG. 14 is a flowchart illustrating collection of a data series within a monitoring and treatment system from a sampling and measuring device in accordance with one embodiment of the present invention; and

FIG. 15 is a flowchart illustrating monitoring and adjusting of a blood glucose analyte level using a monitoring and treatment system in accordance with one embodiment of the present invention.

### DETAILED DESCRIPTION OF THE INVENTION

One aspect of the present invention encompasses a blood analyte sampling and measuring device and its method of use. This method of use can enable medical care personnel to situate a small apparatus on a patient, adjust its sizing to fit the patient properly, affix a replaceable supply cartridge to the apparatus, and leave the apparatus to automatically monitor the patient's blood analytes (such as glucose level) at various intervals as required for an extended period of time without manual intervention.

With use of the various embodiments of the present invention, individual blood analyte measurements may be recorded electronically, displayed to a healthcare provider, or sent directly to a therapeutic control device to adjust infusion or other treatment. In one exemplary embodiment, healthcare personnel need only to change the supply cartridge periodically, thereby enabling several hours of measurements to be taken by the sampling and measuring device without further manual action. Thus, what is currently a manual task may be electromechanically automated by the sampling and measuring techniques of the present invention.

As provided throughout this disclosure, the operation of the present invention is primarily described with relation to one specific type of blood analyte, that of glucose level. Those skilled in the art will recognize numerous other types of blood analyte measurements and treatments may be facilitated through varying techniques and structures without departing from the intended scope of the present invention.

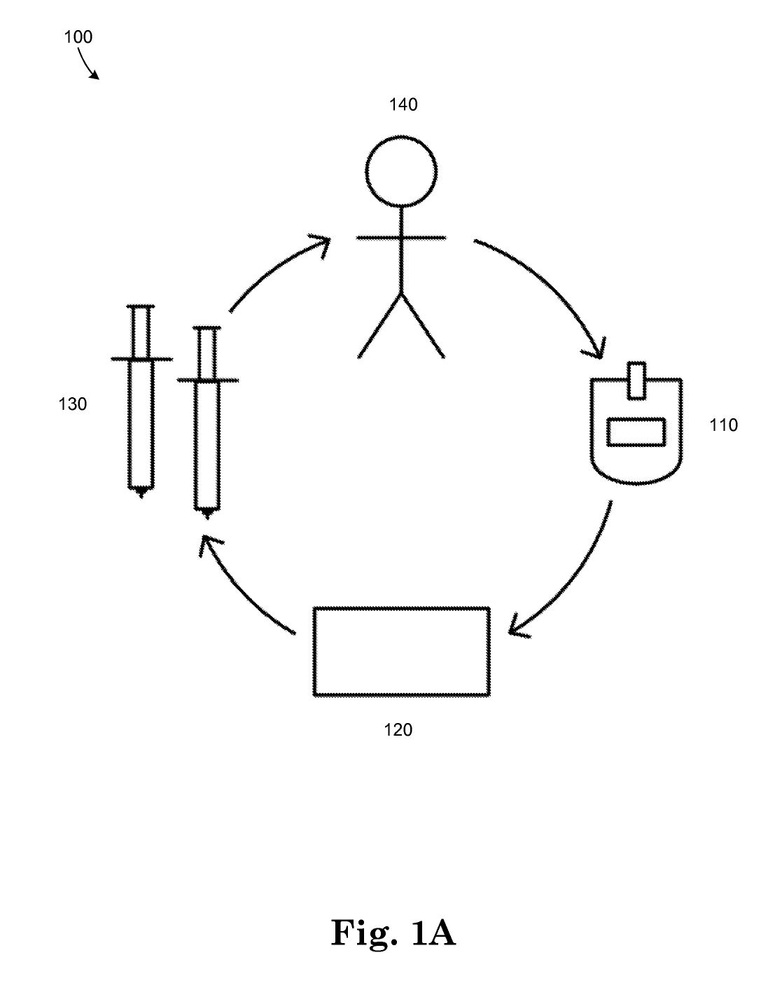

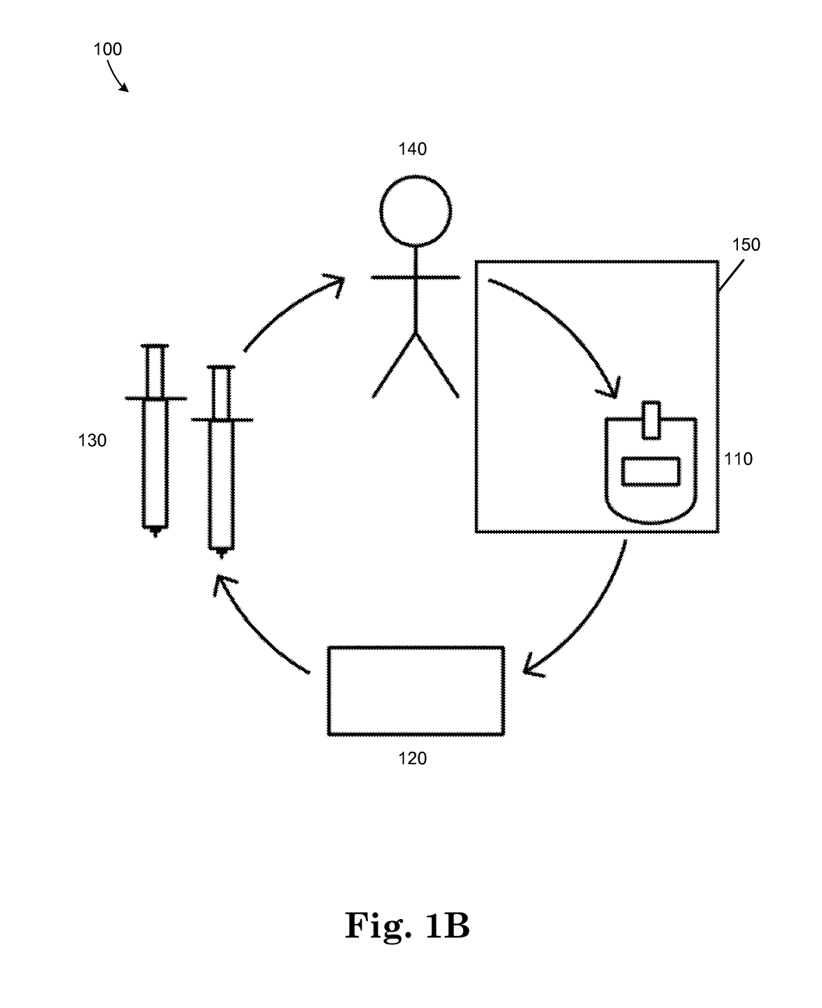

FIG. 1A generally depicts a treatment cycle in which the present invention operates. In step 110, a blood sample is obtained and measured using a sampling and measuring device. In step 120, therapeutic requirements are determined using a suitable modeling and calculation tool. In step 130, treatment is delivered to the patient. In step 140, the patient's response to treatment is reflected in the next sample measurement, and the cycle begins again. By connecting the steps depicted in FIG. 1A, a “closed-loop” system is created. In such a system, one or more physiologic parameters (such as blood glucose) may be accurately monitored, and treatment is calculated and delivered in a continual manner without need for repeated manual action.

The various embodiments of the present invention automate the sampling and measurement portion of this cycle identified as 150 in FIG. 1B. This encompasses the extraction of a blood sample from the patient, the deposition of the sample into a sensing component, and measurement of the blood analyte or analytes. This may additionally encompass relay of measurement results to an external device, such as a treatment controller 120.

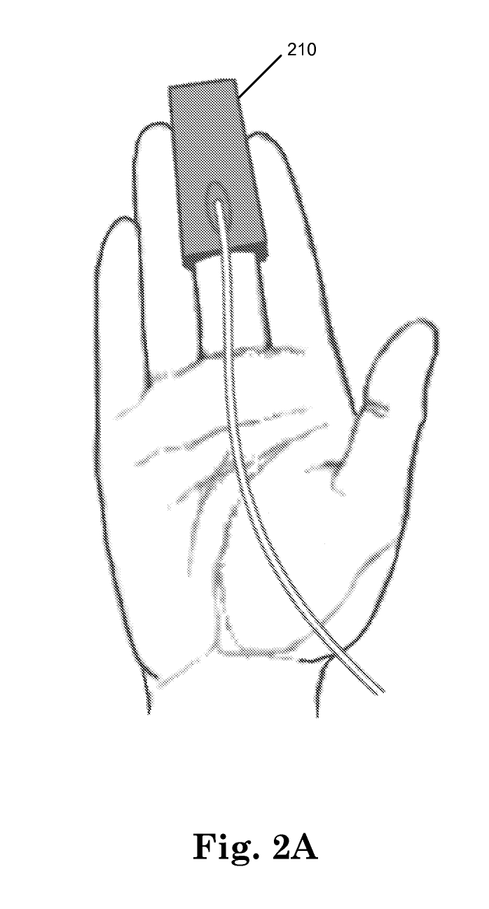

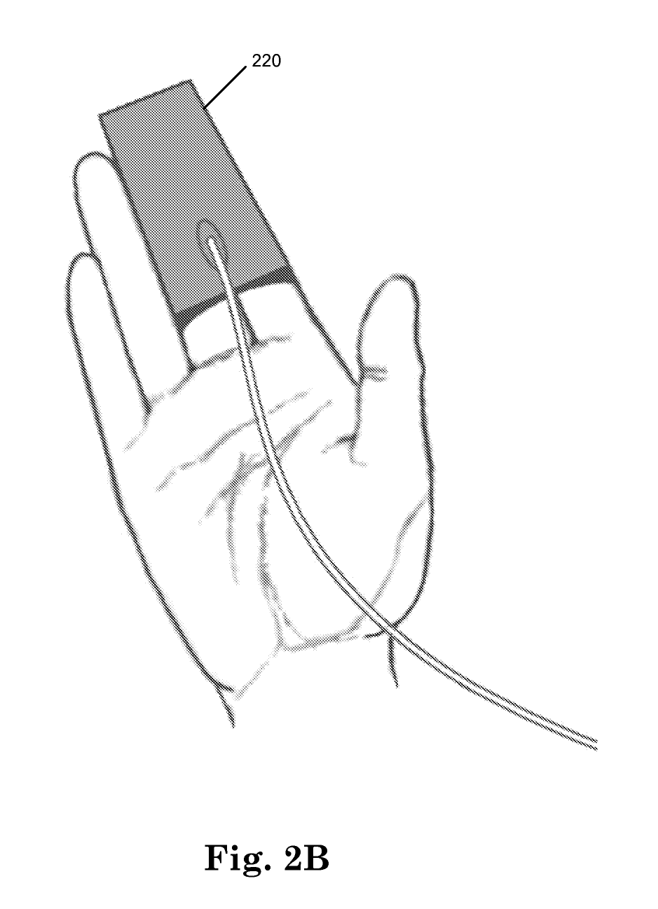

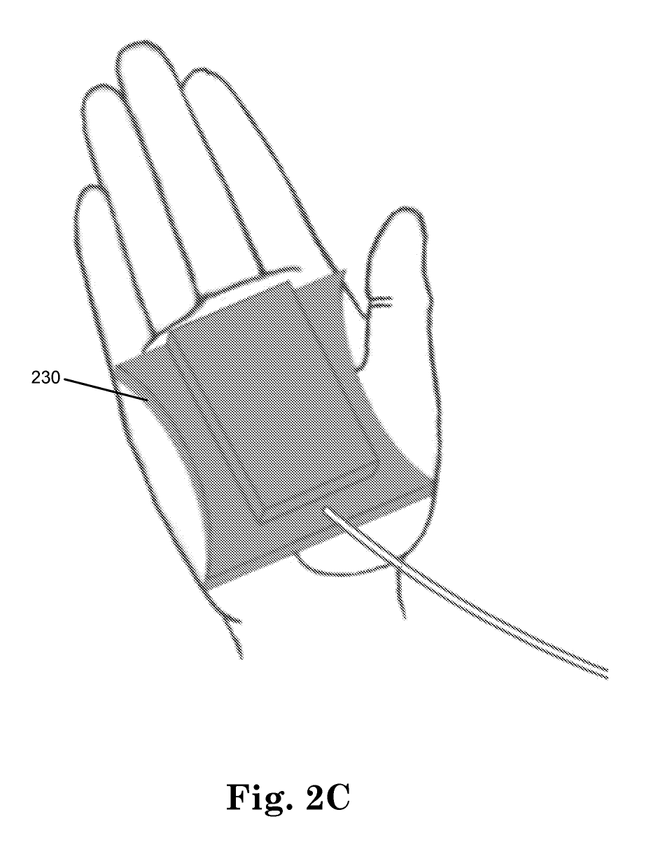

A sampling and measuring device according to one embodiment of the present invention is depicted in FIG. 2A. This representative drawing illustrates one possible embodiment in which capillary blood samples are taken from a single finger of a subject patient through the attachment of the sampling and measuring device 210.

FIG. 2B shows a representative drawing of another possible embodiment of the present invention in which capillary blood samples are taken from multiple fingers of a subject patient. As shown, the sampling and measuring device 220 is configured to be attached to multiple fingers of the patient.

FIG. 2C shows a representative drawing of another possible embodiment of the present invention in which capillary blood samples are taken from a palm of a subject patient. As shown, the sampling and measuring device 230 is configured to be attached to the palm of the subject patient.

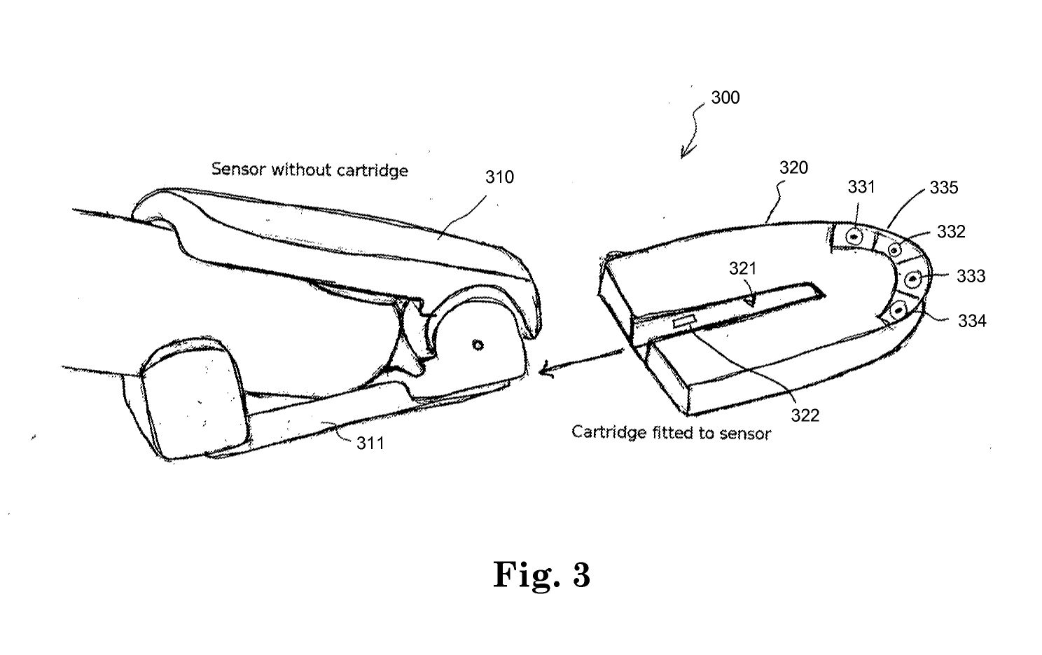

FIG. 3 is a perspective view of one embodiment of a sampling and measuring device 300 used at a single location on a subject patient in accordance with one embodiment of the present invention. The sampling and measuring device 300 generally includes a permanent (i.e., reusable and non-disposable) sensor unit 310 and a replaceable (i.e., non-reusable and disposable) cartridge 320. As shown, the sensor unit 310 is configured to be placed on an extremity of a patient and accept the attachment of the replaceable cartridge 320.

In the embodiment depicted in FIG. 3, the sensor unit 310 contains the electronics, lancet firing mechanism, variable pressure control, and other components designed for repeated and long-lived use on the patient. The sensor unit 310 may be positioned on the patient's finger, palm, forearm, toe, earlobe, or other suitable location in a manner that is easy to attach and remove. As suggested by FIGS. 2A-2C, the form of the sampling and measuring device 300 may differ based on the measuring location, and may be adapted to a variety of body locations. In this embodiment, the sensor unit 310 is a spring-loaded unit having an upper portion and a lower portion operably connected to each other, with the sensor unit configured to be attached onto the tip of the finger and remain attached by application of suitable spring-loaded pressure to the finger. The upper portion and the lower portion may be connected via hinges, flexible tabs, fasteners, flexible joints and such other connectors known to those skilled in the art.

Those skilled in the art would also recognize that the depicted sampling and measuring device 300 may be attached to a finger or other measurement sites of a patient using numerous other means and techniques as known in the art. Further, the sensor unit 310 may also be configured to be fitted or otherwise adjustable to patient physiology, and may account for size and shape of the measuring location used.

The replaceable cartridge 320 may be configured to be fitted into the sensor unit 310. The cartridge 320 may be outfitted with necessary consumable products for testing such as test strips, lancets, anesthetic/analgesic, and absorbent padding. When consumables are exhausted, the cartridge 320 may be replaced as a single unit, mitigating the need to handle consumable items individually.

An exploded view of several of the components that would be found in the replaceable cartridge 320 is also illustrated in FIG. 3. Specifically, the cartridge depicted contains a number of lancets 331, 332, 333, 334 within separate reaction test areas. Each of the reaction areas are in turn separated by separators such as 335. Absorbent padding may also be used in the replaceable cartridge 320 to control bleeding and keep test blood from contaminating unused reaction areas.

The replaceable cartridge 320 may include a slot 321 or similar feature adapted to mate with an attachment means 311, such as a guide rail, on the sensor unit 310. When mated together, one or more electrical contacts 322 on the replaceable cartridge 320 may be positioned adjacent a similar electrical contact on the sensor unit (not depicted) such that the sensor unit 310 and replaceable cartridge 320 are electrically coupled.

Solution may also be applied to the measurement area prior to a measurement or reapplied as necessary using a manual or automated means. In a further embodiment, when situated on a patient, the sampling and measuring device 300 may automatically apply an anesthetic/analgesic solution to the skin around the measurement area.

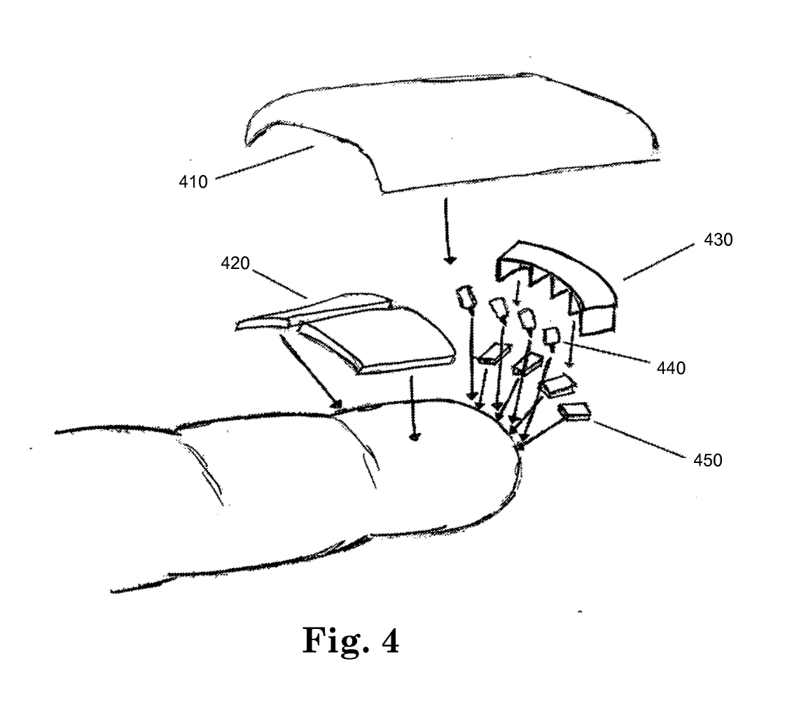

FIG. 4 illustrates a blown-up disassembly of the automated portions used in the sampling and measuring device according to one embodiment of the present invention. The cartridge housing 410 covers each of the disposable components within the replaceable cartridge, such as the lancets 440 and the blood reactive materials 450. The pressure mechanism 420, while part of the sensor unit and not disposable, is shown to illustrate its relation to the reaction test areas.

As illustrated in FIG. 4, a set of lancets 440 is used to pierce the skin to draw blood. The lancets are positioned proximate to a set of blood reactive materials 450, such as glucose test strips. Lancets may be fired automatically on command of the controller within the sampling and measuring device. Lancet penetration depth may be adjusted for all lancets simultaneously or each individually. Proper lancet depth may also be calculated using an external calibrator. Lancets may be arranged on the cartridge such that consecutive measurements may be taken at a maximum distance apart, helping to prevent sample cross-contamination and speed healing of perforated skin.

As further illustrated in FIG. 4, a test area separator 430 may be used to sequester individual measurement sites. In one exemplary embodiment, the walls of the site compartments may be layered in absorbent gauze atop fluid-proof sealant material to prevent cross-contamination of test areas. However, as will be appreciated by those skilled in the art, numerous other sealing means are contemplated and within the intended scope of the present invention such as sealing means including more than two layers.

A pressure inducing mechanism 420, such as a mechanical or pneumatic mechanism, may be utilized to produce pressure gradient patterns to affect blood flow in the measurement area before and/or after sampling. Likewise, variable pressure may be applied as necessary to increase and decrease blood flow. For example, pressure gradient patterns may be applied to increase the blood flow prior to lancet penetration, and pressure may be reversed shortly after measurement to decrease the blood flow. This may ensure that the measurement site has sufficient blood to provide an accurate reading, and minimizes further bleeding once the test has been performed. Force used and area affected in pressure application may be modified for individual patient needs.

In one exemplary embodiment, the pressure inducing mechanism forms one component of the sensor unit. However, the pressure inducing mechanism may alternatively be designed such that it is separate from the sensor unit or is provided by an external source.

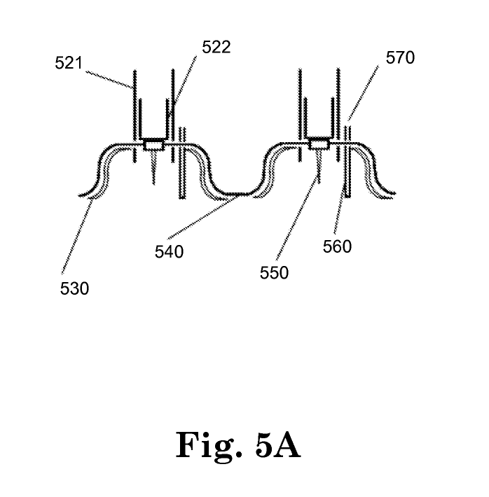

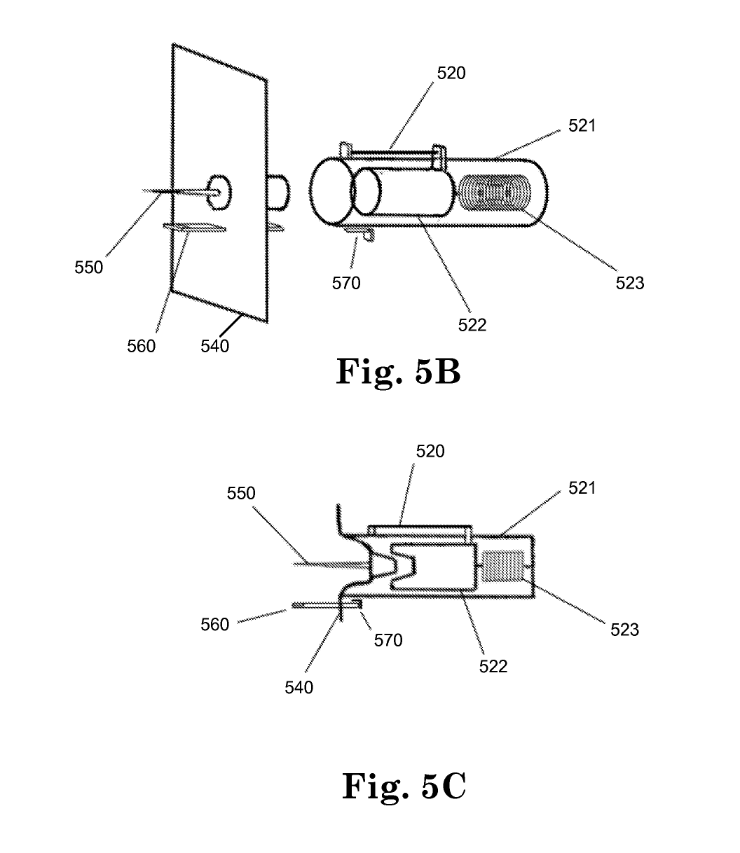

FIG. 5A illustrates a cross section of two adjacent reaction areas used to obtain blood analytes with use of the sampling and measuring device according to one embodiment of the present invention. FIG. 5A depicts the relative positioning of test materials used in the sampling and measuring device including lancets (such as lancet 550 in the second reaction area), test strips (such as test strip 560 in the second reaction area), absorbent padding (such as absorbent pad 530 in the first reaction area), and a flexible barrier membrane 540 above and between both reaction areas. The absorbent material 530 functions to absorb excess blood when a sample is taken.

The permanent, non-disposable materials used in the reaction area include an actuator, in addition to an electronic contact 570 with the test strip 560. In operation, the lancet 550 is activated by an actuator carriage 522 housed within an actuator casing 521. The electronic contact 570 with the disposable test strip 560 then enables measurement of the blood analyte collected within the reaction area.

The membrane 540 forms a flexible barrier that is non-permeable to blood, preventing any blood from one reaction area from contaminating another reaction area. Between reaction areas, the membrane is pressed against the patient's skin to form a seal, aiding in sequestration of the blood sample. The membrane barrier 540 additionally separates the disposable test materials (lancets, test strips, padding) from the non-disposable components (actuators, electronics, permanent casing, etc), preventing fluids from coming in contact with durable parts. The membrane 540 is made of a flexible, tear-resistant material, such as latex or other similar material, allowing movement for lancet actuation while keeping the barrier between disposable and non-disposable components intact.

FIGS. 5B and 5C illustrate additional views of a reaction area used within a sampling and measuring assembly, multiple of which are contained in various embodiments of the present invention. In the depicted assemblies, the disposable test strip 560 is positioned to be connected to electrical contacts 570 in the permanent assembly; and the lancet 550 is positioned to be joined to the actuator carriage 522. In this embodiment, a tension spring 523 keeps the carriage retracted in the casing 521 before and after actuation. During actuation, the actuator 520, a shape metal alloy, contracts in response to an applied electrical stimulus, rapidly pulling the carriage 522 forward and causing the lancet 550 to penetrate the patient's skin to draw blood. The proximity of the test strip 560 to the point of lancing allows blood to flow directly into a reactive chamber on the test strip via capillary action.

Force and travel of lancet actuation may be adjusted as required for an individual patient. For an embodiment in which the actuator is driven by, for example, shape memory material, electromagnetic, or piezoelectric means, lancet force and travel may be adjusted by varying the electrical stimulus applied to the actuator's motive component. Force and travel of lancet actuation may be adjusted for a single reaction area or for multiple reaction areas simultaneously.

As will be appreciated by those skilled in the art, blood may be drawn by capillary action from the point of lancet penetration to a test strip. A chemical reaction will then take place on the test strip in proportion to the concentration of the specific analyte such as glucose present in the blood. Thus, using the glucose example, an electrical charge may be used to determine the magnitude of the test strip reaction and therefore the patient's blood glucose level.

Each reactive test area may have its own electrical sensor, or multiple test strips may be situated on a single circuit, allowing one sensor to service multiple reaction areas. The sensor or sensors may be connected to a data converter which translates the test results into a format suitable for storage, display, relay, or processing by a controller.

As previously mentioned, operation of the sampling and measuring device may be regulated by a programmable controller. This controller may be contained within the non-disposable sensor unit or may be external, such as by linking with an external patient monitor via wired or wireless communications. The controller may be configured to dictate when measurements are taken, and may instruct the sampling and measuring device to retake a measurement if deemed necessary. The sampling and measuring device may report to the controller measurement results and operational status, including how much cartridge supplies have been consumed and how much remain available. In a further embodiment, the programmable controller may be attached or otherwise directly coupled to the sampling and measuring device, to enable fully autonomous operation of the device.

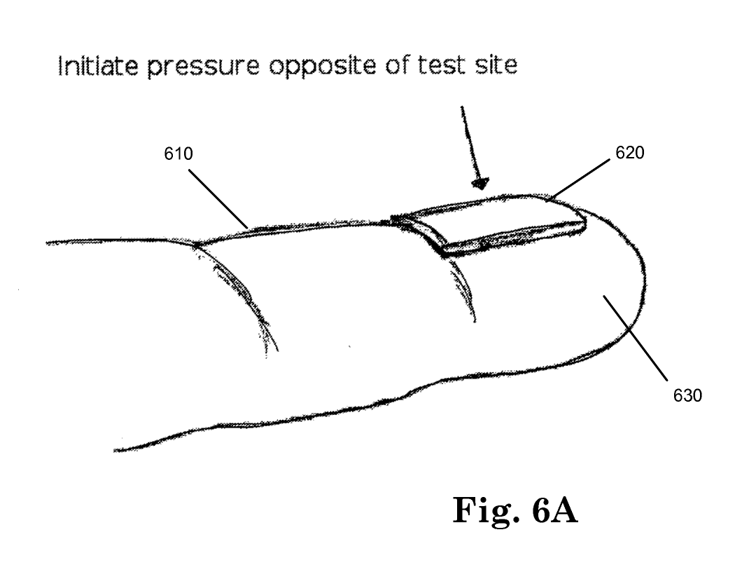

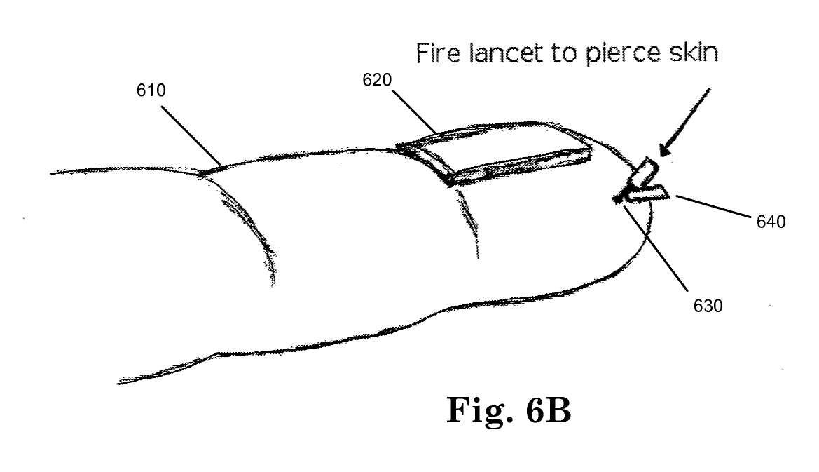

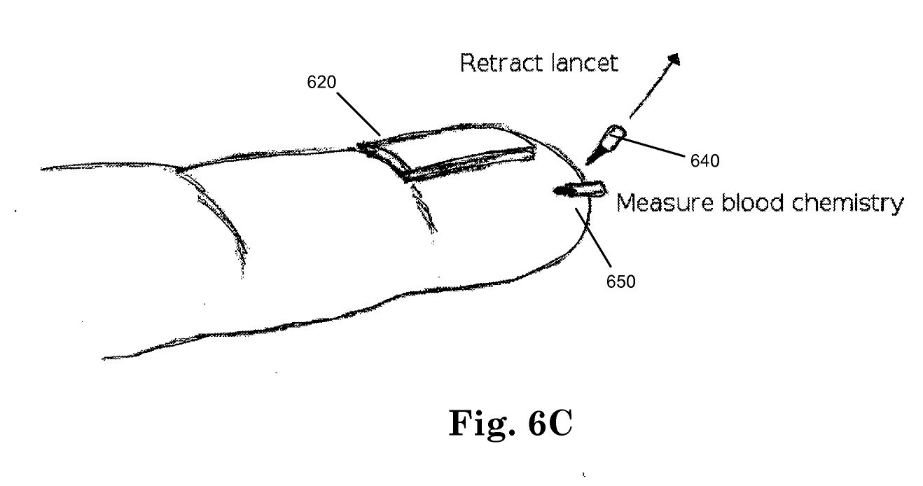

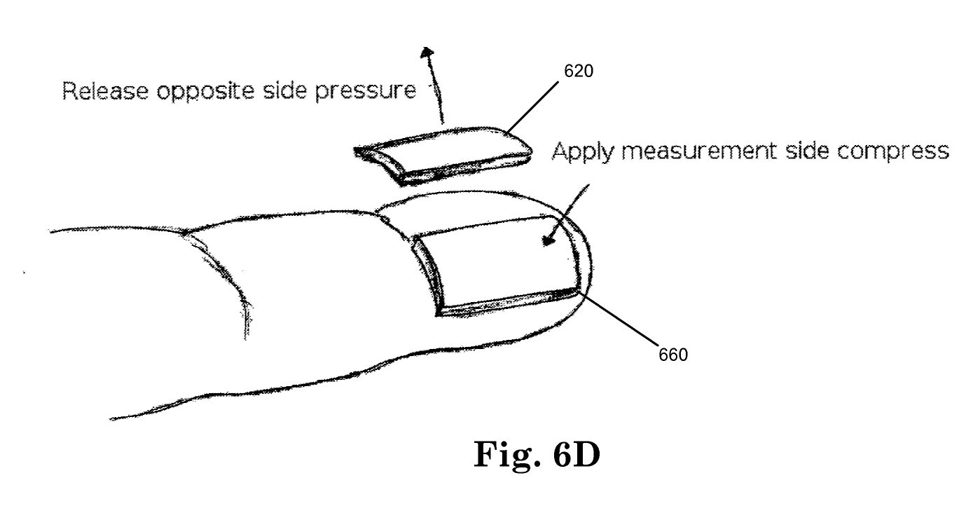

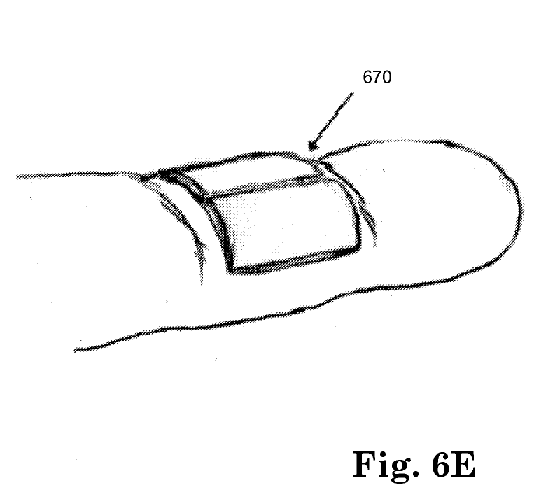

FIGS. 6A-6D illustrate one exemplary method of obtaining blood glucose measurements with the patient monitor by firing a lancet and measuring blood chemistry. Particularly, as shown in FIG. 6A, a pressure pattern may be applied to the tip of a finger 610 adjacent a measurement site 630 with a pressure inducing mechanism 620. This forces more blood to the measurement site area 630 and pushes skin taught at the point of lancing, improving blood sampling.

Next, the lancet 640 is deployed as illustrated in FIG. 6B, piercing the skin to draw blood. As discussed above, in one embodiment lancets may be actuated using shape memory materials, although a variety of approaches could be employed including, for example, electromagnetic, mechanical, chemical, pneumatic, hydraulic, and piezoelectric.

Then, as illustrated in FIG. 6C, the lancet 640 may be retracted and the blood chemistry measured. In particular, as the lancet 640 is withdrawn, blood flows by capillary action onto the chemical test strip 650. The electrochemical reaction produced by the blood on the test chemistry is read electrically by a measurement circuit, and interpreted to determine blood analyte concentration (such as a glucose level). Finally, as illustrated in FIG. 6D, the pressure provided by the pressure inducing mechanism 620 may be released. Optionally, another pressure pattern may be applied to a different location of the finger tip (such as directly on the measurement site) with a second pressure inducing mechanism 660 in order to encourage clotting.

FIG. 6E illustrates an alternative application of pressure used in one embodiment of a sampling and measuring method. As illustrated, a single compress 670 is applied behind the sampling area prior to lancing, and then removed once a blood sample has been obtained. In this embodiment, no second application of pressure is required.

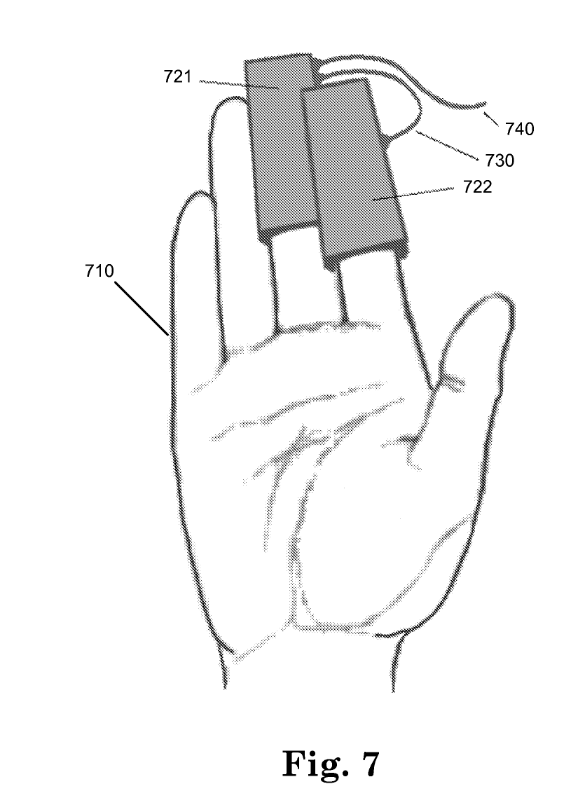

FIG. 7 illustrates the ability of multiple sampling and measuring devices 721 and 722 to be deployed on a single patient hand 710 and linked together electronically to form a chain of sensor devices. These linked devices thereby may expand the number of available measurement sites, and consequently the length of time the sampling and measuring devices can monitor patient condition before requiring resupply of testing materials. In one embodiment, an I²C interface is used to communicate between the multiple sampling and measuring devices 721 and 722 through connection 730. For example, multiple devices can coordinate operations, such as alternating the use of measurement sites to obtain improved results from different patient fingers. Alternatively or in combination, the multiple devices may be monitored and/or controlled through an external interface connection 740.

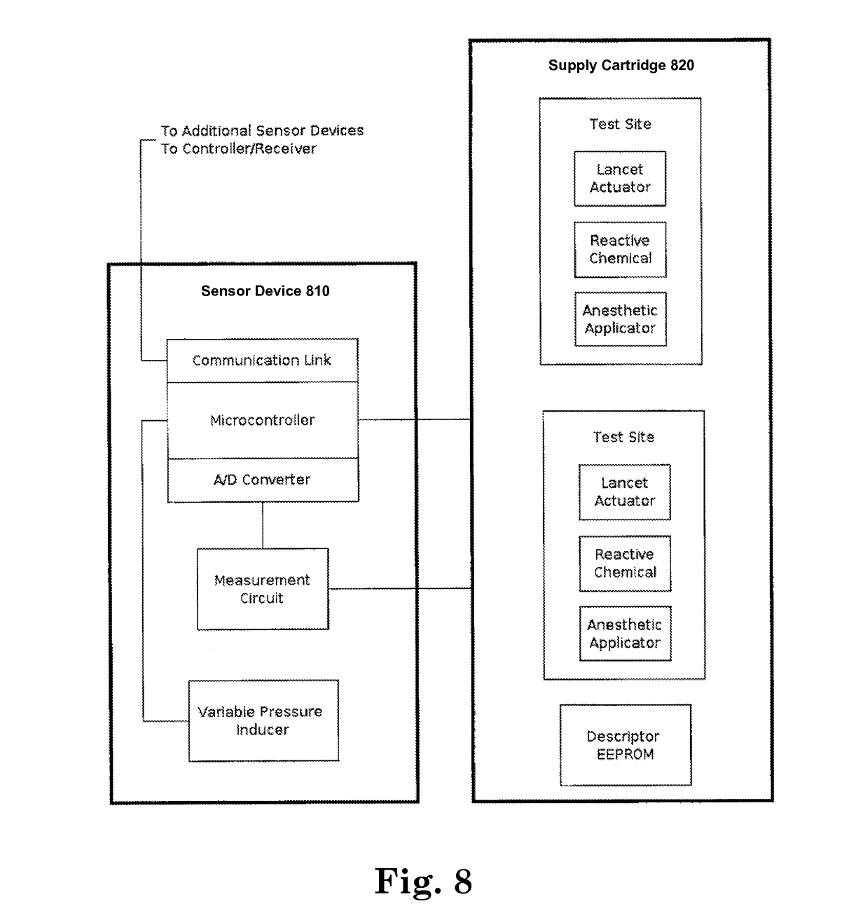

FIG. 8 is a block diagram illustrating the electronic components of a sensor unit assembly 810 and a replaceable supply cartridge 820 deployed within a sampling and measuring device in accordance with one embodiment of the present invention. As generally illustrated in FIG. 8, the primary electronic components used for initiating and controlling the sampling and measuring operations may reside in the sensor assembly 810. These components may include the microcontroller, analog-to-digital converter and measurement circuit, and variable pressure mechanism.

In one exemplary embodiment of the present invention, the sensor unit 810 microcontroller is configured to receive a command via a communication link to commence with the blood analyte testing. In this embodiment, the sampling and measuring device operates as a “slave” to an external controller, conducting a sampling and measuring operation only when instructed to by the external controller, and communicating the results of the sampling and measuring to the external controller. However, the control of the actuator, any reactive chemical or anesthetic, and the actual measurement of the blood analyte from the measurement site occurs through microcontroller control and other logic internal to the sensor unit 810.

Once the blood analyte measurements are obtained and processed within the sensor unit 810, it is then communicated via the communication link to the external source or controller. Those skilled in the art would recognize that additional functionality could be added to the sensor unit 810 to enable fully autonomous, non-slave operation of the sampling and measuring device.

In one exemplary embodiment, the variable pressure inducer within the sensor unit may be a two-sided rocker mechanism, although numerous other pneumatic, hydraulic, mechanical, or other means are also contemplated. The communications link to additional sensor devices or an external controller/receiver may utilize, for example, a wired USB connection. Alternatively, any other suitable bus or communication may be used in conjunction with the sampling and measuring device including RS-232 serial, Bluetooth, and 802.11 wireless configurations.

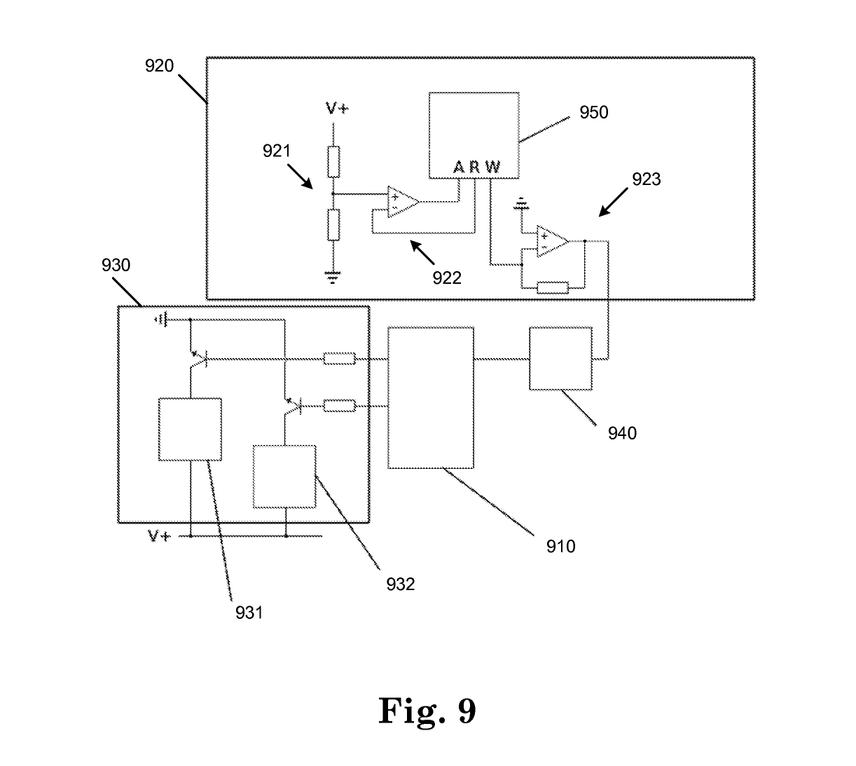

FIG. 9 is a high-level circuit diagram depicting the electrical components which control sampling and measurement at individual reactive test area within one embodiment of a patient sampling and measuring device. A set of circuits (measurement circuit 920 and actuator circuit 930) used for control of a single reactive test area are shown. One embodiment of the present invention incorporates multiple such circuits, one for each reactive test area in the device, all connecting to the same sensor unit microcontroller 910. For the single test area 930, a measurement circuit 920 and an actuator circuit 930 are each connected to the microcontroller. In one embodiment, individual lancet actuators exist for each test area, while a single pressure actuator services multiple test areas.

The measurement circuit 920 comprises a set of connections to a test strip 950, accompanied by use of a voltage divider 921, a voltage follower 922, and a current-to-voltage converter 923. The measurement circuit is connected to the microcontroller through an analog-to-digital converter 940. The actuator circuit 930 comprises connections to a pressure actuator 931 and a lancet actuator 932, connected for electronic control by the microcontroller 910.

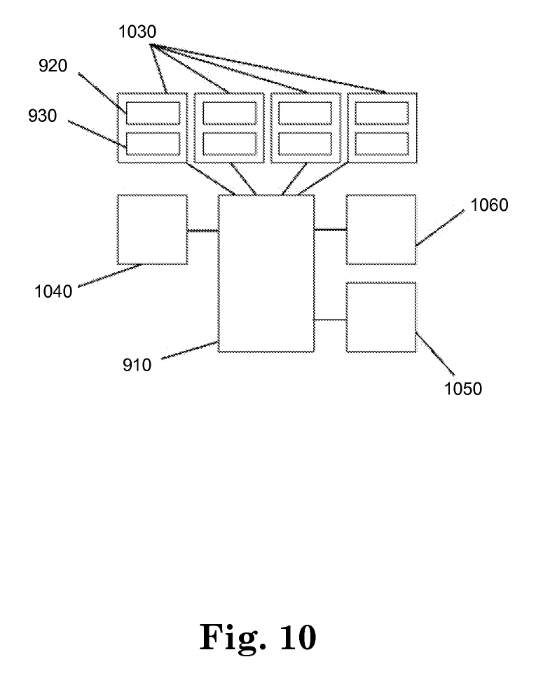

FIG. 10 is a circuit diagram depicting the microcontroller 910 and the subcircuits connected to it. An example of the individual test area circuits 1030 is shown in more detail in FIG. 9, and contains the measurement circuit 920 and actuator circuit 930. The cartridge detection/EEPROM circuit 1040 allows the microcontroller 910 to determine electrically when a supply cartridge is present, in addition to reading information about the cartridge including the quantity and state of the supplies it contains. The sensor interconnect 1050 is an inter-integrated circuit bus connection allowing multiple sampling and measuring devices (and/or additional device test areas) to be linked together. The remote interconnect 1060 is a communications interface using, for example, USB or RS-232 serial communications to interface with a remote device, accepting commands, and reporting measurement results and status.

As previously suggested, the disposable supply cartridge may contain all consumable testing supplies, including lancets and glucose reactive tests. In use, the cartridge may be affixed to the sensor unit of the sampling and monitoring device, establishing several electrical connections between the two and giving the sensor unit access to all cartridge resources. In a further embodiment, the replaceable supply cartridge may include a descriptor memory chip (EEPROM) which may allow cartridge attributes to be queried by the microcontroller. Attributes may include available test count (which may be decremented as test areas are used), and test strip chemistry characteristics.

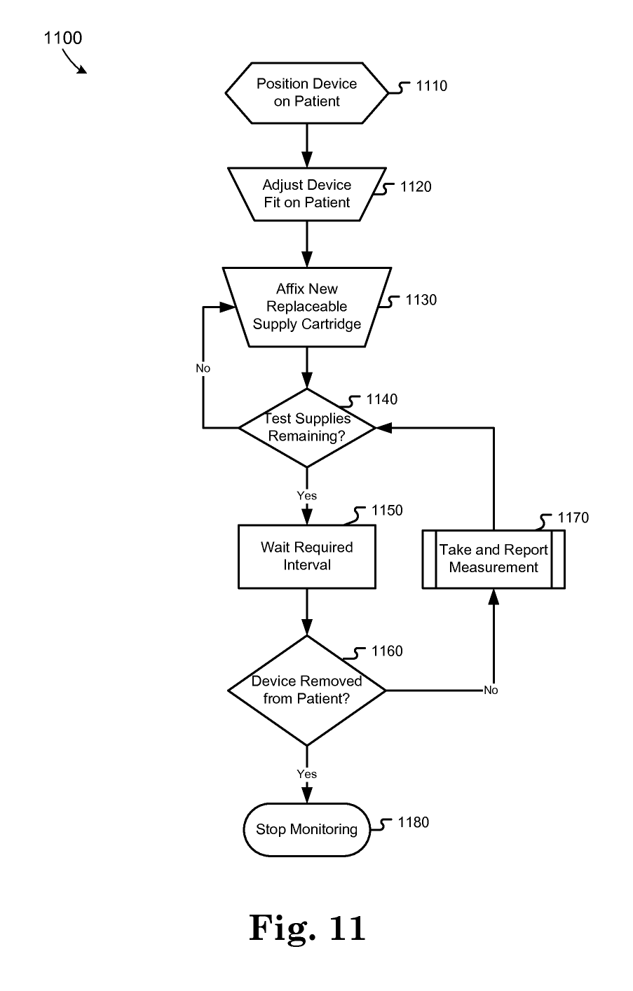

FIG. 11 is a flowchart illustrating one exemplary embodiment of a method 1100 for deploying an automated blood analyte sampling and measuring device on a patient in accordance with the present invention. As illustrated in FIG. 11, method 1100 begins at step 1110 by positioning the patient monitor on a desired location on the patient. The patient monitor may be, for example, similar to the patient monitor described with reference to FIG. 2A and attached to a patient's finger. However, patient monitors that are adapted for positioning at locations including the patient's palm, multiple fingers, forearm, toe, earlobe, or any other suitable measuring location are contemplated within the scope of the present invention.

Method 1100 continues at step 1120 where the patient monitor device (i.e., the sampling and measuring device) may be adjusted to fit the specific size and contours of the patient physiology at the measuring location. This adjustability allows the patient monitor device to be tailored to variations in the size and shape of measuring locations of different patients. As a result, the patient monitor device may be “universal” such that one device design may be used on substantially all patients.

Next, in step 1130, a new supply cartridge is inserted or otherwise affixed to the sensor unit portion of the patient monitor device. Once the cartridge is attached to the sensor unit portion of the patient monitor device, the consumable products located within the cartridge are tested in step 1140 to ensure there is a sufficient amount of the products remaining. If it is determined that there is not a sufficient amount of the products remaining, the method returns back to step 1130 where the user must insert a new supply cartridge into the sensor unit. However, if it is determined that there is a sufficient amount of the consumable products remaining in the cartridge, then the method continues at step 1150 where a predetermined, required time interval is monitored prior to taking any measurements. The predetermined, required time interval may be a configurable parameter selectable by the user or provided by an external control system. Thus, for example, the required time interval in step 1150 may be any amount of time greater than or equal to zero seconds. As those skilled in the art will appreciate, when the required time interval is set to zero seconds, step 1150 is essentially “skipped” such that the method moves almost immediately from step 1140 to step 1160.

Once the required time interval has elapsed, the method continues in step 1160 with determining whether the patient monitor device has been removed from the patient. If it is determined that, for any reason, the patient monitor device has been removed from the patient, the method continues to step 1180 wherein the monitoring process is stopped. Additionally, an external monitoring system may be alerted to the removed monitor. However, if it is determined that the required time interval has elapsed and the monitor remains positioned on the patient, then the method continues at step 1170 where blood analyte measurements are taken and reported to the user, patient, or to an external system.

Once one or more blood glucose measurements are taken and reported in step 1170, the method returns to step 1140 wherein the consumable products located within the cartridge are tested to ensure a sufficient amount of the products still remains in the cartridge. If a sufficient amount of consumable products is not found in the cartridge, such as when all consumable products and test areas have been utilized, then the method returns to step 1130 where the user is required to insert a new cartridge into the sensor unit of the patient monitor. However, if a sufficient amount of the consumable products still remains within the cartridge, then the method continues with taking and reporting additional measurements, again repeating the process as long as the monitor has not been removed from the patient.

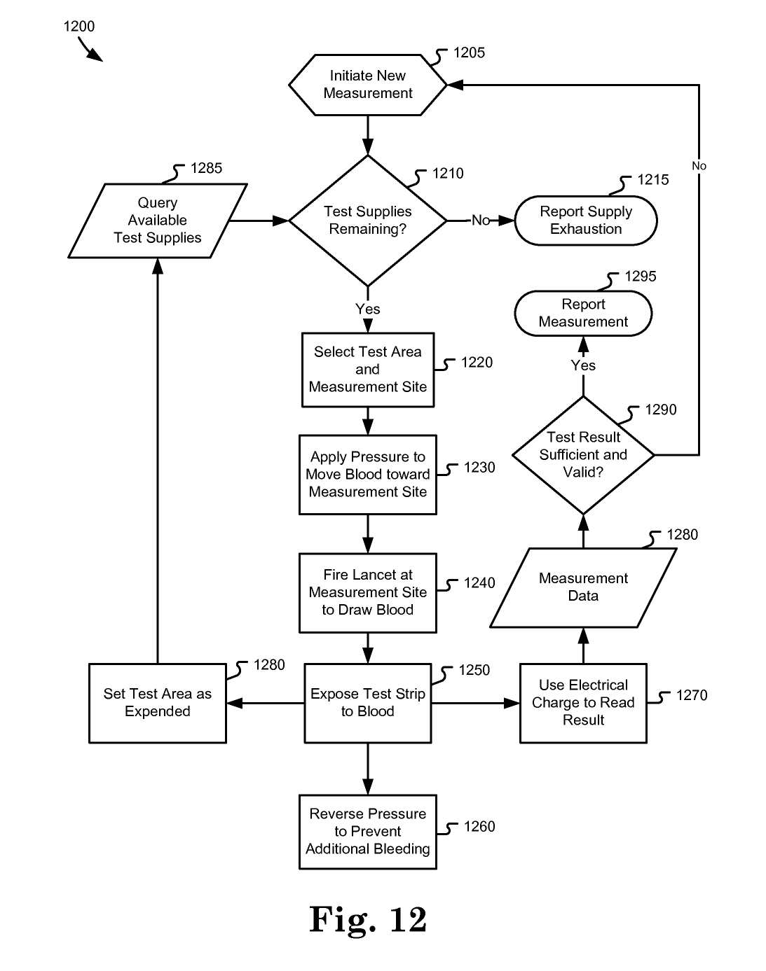

FIG. 12 is a flowchart illustrating one exemplary embodiment of a method 1200 for obtaining blood analyte measurements with a sampling and measuring device in accordance with the present invention. As will be appreciated by those skilled in the art, the steps in FIG. 12 represent a logical counterpart to the physical process illustrations shown in FIGS. 6A-6D. As illustrated in FIG. 12, method 1200 begins at step 1205 by initiating a new measurement. The method continues at step 1210 where a determination is made whether any consumable products and/or test areas remain within the cartridge.

If it is determined that there is not a sufficient amount of the consumable products remaining within the cartridge, then the method proceeds to step 1215 where exhaustion of the supply may be reported to the user or an external system. The supply exhaustion may be reported by, for example, a signal sent from the patient monitor to an external controller via a communications interface. Alternatively, if it is determined that a sufficient amount of consumable products remains, then the method continues at step 1220 where a specific test reaction area is selected for sampling of a specific measurement site on the patient. Once the specific test area has been selected, pressure is applied around the measurement location in step 1230 in order to induce blood to flow toward the specific measurement site. In one embodiment, pressure may be applied via an inflatable mechanism structured to produce pressure gradient patterns to cause an increase in blood flow at the measurement site as previously described.

After blood flow has been increased in the area surrounding the specific measurement site, method 1200 continues at step 1240 where a lancet is “fired” or otherwise deployed to the skin of the measurement site in order to draw blood for use by the patient monitor device. Next, in step 1250, a test strip within the selected test area is exposed to the blood previously drawn by the lancet. The pressure applied to increase blood flow at the measurement site is thereafter reversed in step 1260 so as to prevent additional bleeding.

The process continues at step 1280 where the specific test area and measurement site that was selected may be indicated as “expended.” The effect of indicating a specific measurement site as expended may be that when subsequent measurements are initiated, different sites may be selected such that a measurement is not repeatedly taken in the exact same location on the patient. In one embodiment, the method in accordance with the present invention may be configured to take measurements at a plurality of locations such that a measurement is not repeated at a particular location until measurements have been taken at all other available locations.

Next, in step 1270, an electrical charge is generated in order to read the electrochemical result on the test strip. This may be accomplished by determining the magnitude of the test strip chemical reaction as previously discussed. Thereafter, in step 1280, the result is translated to human readable measurement data with a data converter (such as an analog to digital converter). The result is then validated in step 1290. If it is determined that the measurement is sufficient and valid, then the process continues at step 1295 where the measurement is reported, such as on a display of the sampling and measurement device, or via a communication to an external controller or monitoring system. If the measurement is not sufficient or not valid, then the process continues back at step 1205 where another new measurement is initiated.

As will be appreciated by those skilled in the art, the processes depicted in FIGS. 11 and 12 are only exemplary embodiments of methods for obtaining blood analyte measurements in accordance with the present invention. Thus, the order, number, and content of the illustrated steps may be altered without departing from the intended scope of the present invention. Furthermore, as will also be appreciated by those skilled in the art, although each of the illustrated processes are used to collect a single test result, the process may be modified to be repeated in order to obtain any number of results over a specified period of time. For example, in one alternative embodiment, a step may be added that monitors the number of measurements taken and/or the amount of time that has elapsed since measurement process began. In this way, a limit may be placed on the number of measurements taken and/or the amount of monitoring time.

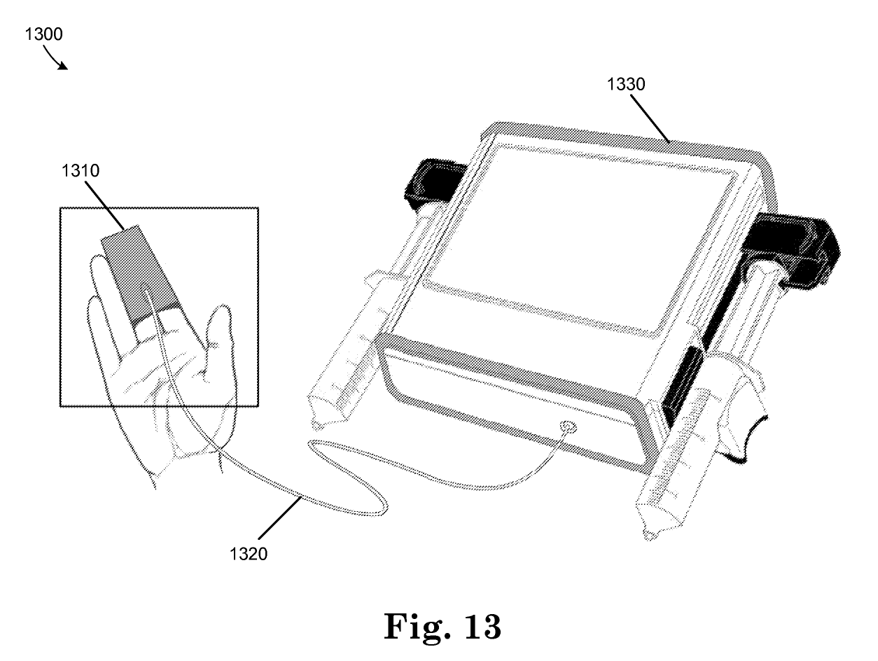

A further embodiment of the present invention involves the combination of the presently disclosed blood analyte sampling and measuring device with various features of monitoring and treatment systems. The use of monitoring and treatment systems enables full or near-full automation of the cycle involving measurement, monitoring, and treatment for specific levels of a blood analyte. Further, use of the presently disclosed sampling and measuring device with a monitoring and treatment system may encompass the relay of measurement results from the sampling and measuring device to numerous external devices, such as a treatment controller, as suggested in FIG. 13.

As is depicted in the system of FIG. 13, a blood analyte sampling and measuring device 1310 is connected via a communications interface 1320 to an external treatment controller 1330. As one skilled in the art would recognize, this treatment controller may perform a variety of functions in a clinical or hospital setting, such as automatically delivering insulin and/or glucose to the patient based on the blood analyte measurements obtained from the sampling and measuring device 1310.

In other embodiments of the present invention, the presently disclosed blood analyte sampling and measuring device and methods of its use may be interfaced with other types of external monitoring devices, treatment control devices, or monitoring and treatment systems. For example, the sampling and measuring device may be used in conjunction with the system and method entitled “Balanced Physiological Monitoring and Treatment System,” disclosed in U.S. patent application Ser. No. 11/816,821, filed Aug. 21, 2007, which is herein incorporated by reference in its entirety.

Monitoring and treatment systems enable the automated regulation of a patient's physiological condition by monitoring at least one physiological parameter, in this case, a blood analyte. In addition to the presently disclosed sampling and measuring device, an example monitoring and treatment system may include an intelligent control device and a multi-channel delivery device for providing controlled intravenous delivery of medications that affect the physiological condition being controlled (namely, the blood analyte level). Control logic in the intelligent control device is derived by an algorithm based on model predictive control. The control logic may include mathematically modeled systems, empirical data systems or a combination thereof. Further, the system may be networked to provide centralized data storage and archival of system information as well as data export and query capabilities that can be used for patient file management, health care facility management and medical research.

The various embodiments of monitoring and treatment systems typically provide a delivery mechanism. This delivery mechanism may include a plurality of pumps for delivering infusion or other treatment to the patient, such as the infusion of insulin to correct an improper level of blood glucose. As those skilled in the art will appreciate, alternate embodiments may include additional pumps and control valves, continuous and/or intermittent pumps, and the administration of fluids that may vary by the time of day, by interval, and by direct or indirect response to the blood analyte monitoring results. Further, a single mechanism may be used in a system configured to monitor and regulate a single or numerous types of blood analytes, in addition to monitoring and treating other physiological parameters and conditions.

Multiple delivery mechanisms further may be used individually or in combination to provide delivery of various medications in monitoring and treatment systems. For example, a single delivery mechanism may control delivery of one or more medications to a patient as determined by a monitoring and treatment system controller and its interaction with a blood analyte sampling and measuring device, or multiple delivery mechanisms may be used with one sampling and measuring device. The monitoring and treatment system controller further may be provided with adaptive logic for gradual, optimized, stabilization of an improper blood analyte level or related physiological condition. Furthermore, the controller may include an output to the delivery mechanism to thereby control the rate of flow of the medication to patient to maintain the patient's blood analyte level and other physiological parameters within a defined range. The monitoring and treatment system controller may accept as input data point information from the blood analyte sampling and measuring device providing the blood analyte measurement in the patient.

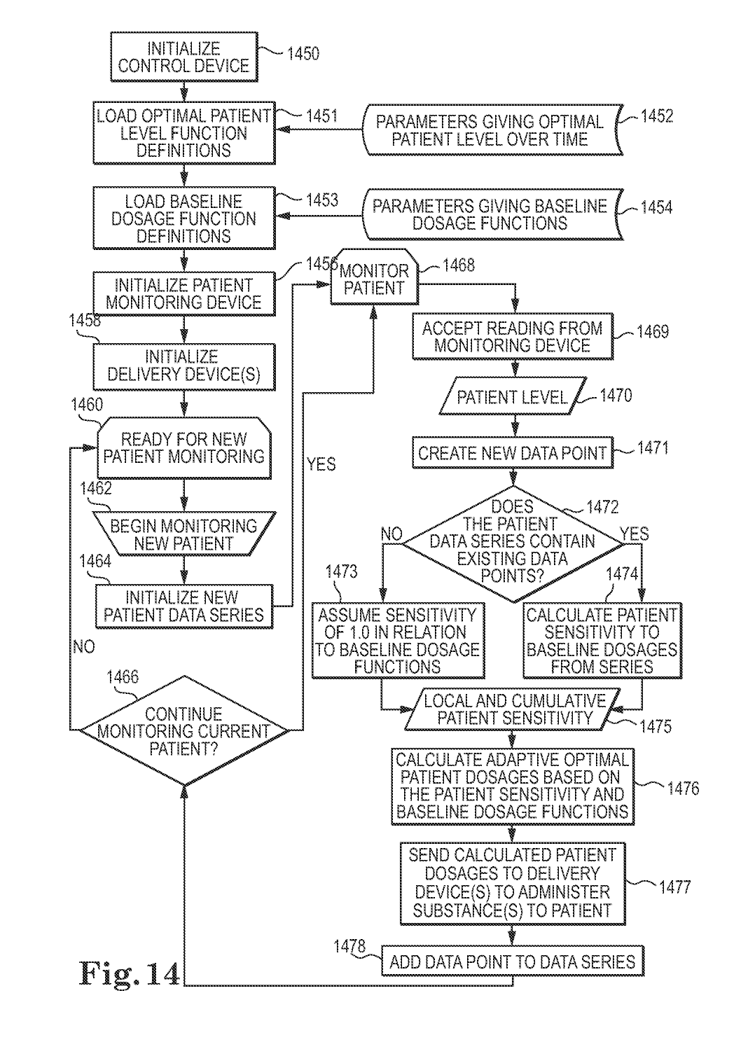

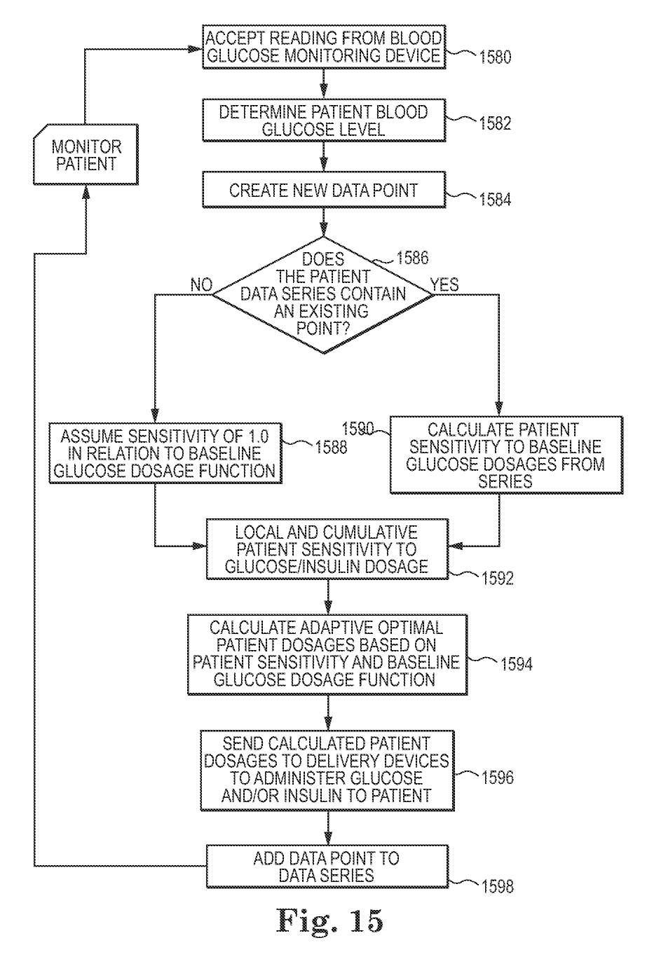

As additional examples of data collection and treatment activities performed within monitoring and treatment systems, FIGS. 14 and 15 illustrate an example interface between treatment control and delivery devices, and a monitoring device such as the blood analyte sampling and measuring device described in the present disclosure. For example, as shown in FIG. 14, a data series may be collected from a monitored patient, enabling the calculation and delivery of optimal patient dosages to change a blood analyte related condition. Likewise, as shown in FIG. 15, a sampling and measuring device configured to read blood glucose can be monitored within the monitoring and treatment system to deliver glucose and/or insulin to a patient throughout a monitored data series.

Those skilled in the art will appreciate that monitoring and treatment systems and devices used in combination with the embodiments of the present invention may include stationary systems used in intensive care units or emergency rooms in hospitals. Alternatively, the systems and devices may comprise portable units for use in other situations, such as in an ambulance or at a person's home.

In further embodiments, monitoring and treatment systems may be integrated with a network for remote monitoring, management, and control of delivery devices and/or the sampling and measuring device. For example, a networked monitoring and treatment system may provide centralized data storage and archival of system information, patient information, blood analyte measurements, and calculation and administered dosage information. Additionally, a networked monitoring and treatment system may provide for information export and query capabilities that may be used for external patient file management, health care facility management, and medical research.

As will be understood by one skilled in the art, various aspects of the present invention may be embodied as a system, apparatus, method, or computer program product. Accordingly, inventive aspects of the present invention may be embodied through use of hardware, software (including firmware, embedded software, etc.), or a combination therein. Furthermore, aspects of the present invention may include a computer program product embodied in one or more computer readable storage medium(s) having computer readable program code embodied thereon.

Code for carrying out operations for aspects of the present invention may be written in any combination of one or more programming languages, including an object oriented programming language such as Java, C#, C++ or the like, conventional procedural programming languages, such as the “C” programming language, or languages configured for use in embedded hardware and other electronics. Further, the various components of the invention described in the drawings and the disclosure above may be implemented by executable program code or other forms of electronic and computer program instructions. These electronic and computer program instructions may be provided to a processor or microprocessor of a general purpose computer, special purpose computer, standalone electronic device, or other data processing apparatus to produce a particular machine, such that the instructions, which execute via a processor or other data processing apparatus, create suitable means for implementing the functions/acts specified in the present drawings and disclosure.

As would also be understood by one skilled in the art, network connections to the previously described devices and systems may be configured to occur through local area networks and networks accessible via the Internet and/or through an Internet service provider. Likewise, network connections may be established in wired or wireless forms, to enable connection with a detached device such as a handheld, laptop, tablet, or other mobile device. For example, a suitable monitoring and control system may be accessible remotely by a third party user via a network connection.

Further, the external controllers, devices, and systems described in the present disclosure may comprise general and special purpose computing systems, which may include various combinations of memory, primary and secondary storage devices (including non-volatile data storage), processors, human interface devices, display devices, and output devices. Such memory may include random access memory (RAM), flash, or similar types of memory, configured to store one or more applications, including but not limited to system software and applications for execution by a processor.

Examples of external computing machines which may interact with the presently disclosed sampling and measuring device and/or monitoring and treatment systems may include personal computers, laptop computers, notebook computers, netbook computers, network computers, mobile computing devices, Internet appliances, or similar processor-controlled devices. Those skilled in the art would also recognize that the previously described systems and devices may also be configured for control and monitoring via a web server, web service, or other Internet-driven interface.

Although various representative embodiments of this invention have been described above with a certain degree of particularity, those skilled in the art could make numerous alterations to the disclosed embodiments without departing from the spirit or scope of the inventive subject matter set forth in the specification and claims.

What is claimed is:

1. An automated device for sampling and measuring blood analytes, comprising:
a sensor unit structured to be positioned on a patient body, the sensor unit including electronic circuitry, lancet firing means, and variable pressure control means; and
a replaceable cartridge having a plurality of consumable products disposed therein, the consumable products including one or more lancets and one or more test strips for measuring a blood analyte of the patient;
wherein the replaceable cartridge and the sensor unit are structured to temporarily mate with one another via an attachment means such that the replaceable cartridge is removable from the sensor unit;
wherein the electronic circuitry enables automated blood extraction and analysis of a blood analyte from the patient body iteratively over time without need for manual intervention; and
wherein the automated blood extraction and analysis is performed through electronically controlled use of the variable pressure control means, the lancet firing means, the one or more lancets, and the one or more blood test strips.

2. The automated device of claim 1, wherein the sensor unit comprises an upper portion and a lower portion operably connected thereto.

3. The automated device of claim 2, wherein the upper portion and the lower portion of the sensor unit is configured to be positioned around a finger of the patient, to obtain a blood analyte measurement from a measurement site at a tip of the finger of the patient.

4. The automated device of claim 1, wherein the sensor unit is adjustable during positioning of the sensor unit upon the patient body.

5. The automated device of claim 1, wherein the lancet firing means provided by the sensor unit comprises an actuator, a tension spring, and an actuator carriage enclosed within an actuator casing.

6. The automated device of claim 1, wherein the lancet firing means includes an actuator, and wherein depth of lancet actuation is adjustable for the one or more lancets individually or in combination.

7. The automated device of claim 1, wherein the automated device automatically applies anesthetic or analgesic solution to skin proximate to a measurement site of the patient prior to firing of the one or more lancets.

8. The automated device of claim 1, wherein the sensor unit further comprises an electronic contact used to read a test strip provided by the replaceable cartridge during automated blood analysis.

9. The automated device of claim 1, wherein the replaceable cartridge provides a plurality of reaction areas, each reaction area containing a lancet and a test strip deployed for obtaining a single blood sample.

10. The automated device of claim 9, further comprising a barrier structure comprising a combination of an absorbent material and a material non-permeable to blood arranged around each reaction area, and wherein the barrier structure separates the consumable products of the replaceable cartridge from the sensor unit.

11. The automated device of claim 1, wherein the replaceable cartridge contains electronic circuitry to provide status of the consumable products.

12. The automated device of claim 1, wherein the blood analyte is selected from the group consisting of triglycerides, total cholesterol, HDL-cholesterol, fibrinogen, hemoglobin, ferritin, and glucose.

13. The automated device of claim 1, wherein the blood analyte is glucose, and wherein the one or more test strips comprise one or more glucose test strips.

14. The automated device of claim 1, wherein the electronic circuitry is connected to a remote controller.

15. The automated device of claim 1, wherein the electronic circuitry includes a microcontroller.

16. The automated device of claim 15, wherein a set of electronic instructions causes the microcontroller to initiate the sequence iteratively within the electronic circuitry over a period of time.

17. The automated device of claim 15, wherein a remote controller causes the microcontroller to initiate the sequence within the electronic circuitry upon request.

18. An automated device for sampling and measuring a blood analyte of a patient, comprising:
a sensor unit structured to be positioned adjacent a measurement site of a patient, the sensor unit including an upper portion and a lower portion operably connected thereto, the sensor unit including an lancet firing means; and
a replaceable cartridge in mating relationship with the sensor unit via an attachment means such that the replaceable cartridge is removable from the sensor unit, the replaceable cartridge housing a plurality of consumable products disposed therein for producing a blood sample, the consumable products including one or more lancets and one or more test strips for measuring the blood analyte of the patient;
a microcontroller and electronic circuitry operably coupled to the sensor unit and capable of controlling use of the lancets and test strips relative to the measurement site; and
a set of electronic instructions executable by the microcontroller such that upon execution, the electronic instructions causes the microcontroller to initiate a sequence comprising selecting a lancet for deployment at a measurement site, firing the lancet to obtain a blood sample from the measurement site, and collecting a blood sample from the measurement site onto a test strip;
wherein the microcontroller receives inputs from the test strip to determine the blood analyte and further wherein the electronic instructions causes the microcontroller to initiate the sequence without the need for manual intervention.

19. The automated device of claim 18, wherein the sensor unit further contains the microcontroller, the electronic circuitry, and a variable pressure control means.

20. The automated device of claim 18, wherein the sensor unit is structured to be positioned around the finger of a patient.

21. The automated device of claim 18, wherein the sensor unit is adjustable during positioning of the sensor unit adjacent the measurement site of the patient.

22. The automated device of claim 18, wherein the lancet firing means provided by the sensor unit comprises an actuator, a tension spring, and an actuator carriage enclosed within an actuator casing.

23. The automated device of claim 18, wherein the lancet firing means includes an actuator, and wherein depth of lancet actuation is adjustable for the one or more lancets individually or in combination.

24. The automated device of claim 18, wherein the automated device automatically applies anesthetic or analgesic solution to skin proximate to the measurement site prior to firing the lancet.

25. The automated device of claim 18, wherein the sensor unit further comprises an electronic contact used to read the test strip provided by the replaceable cartridge during automated blood analysis.

26. The automated device of claim 18, wherein the replaceable cartridge provides a plurality of reaction areas, each reaction area containing a lancet and a test strip deployed for obtaining a single blood sample.

27. The automated device of claim 26, further comprising a barrier structure comprising a combination of an absorbent material and a material non-permeable to blood arranged around each reaction area, and wherein the barrier structure separates the consumable products of the replaceable cartridge from the sensor unit.

28. The automated device of claim 18, wherein the replaceable cartridge contains electronic circuitry to provide status of the consumable products.

29. The automated device of claim 18, wherein the blood analyte is selected from the group consisting of triglycerides, total cholesterol, HDL-cholesterol, fibrinogen, hemoglobin, ferritin, and glucose.

30. The automated device of claim 18, wherein the blood analyte is glucose, and wherein the one or more test strips comprise one or more glucose test strips.

31. The automated device of claim 18, wherein the microcontroller is connected via a communications interface to a remote controller.

32. The automated device of claim 31, wherein the remote controller causes the microcontroller to initiate execution of the sequence within the electronic circuitry upon request.

33. An automated system for monitoring blood analytes of a patient, comprising:
a sampling and measurement device structured to be positioned adjacent a measurement site of a patient, the device housing a replaceable supply of consumable products including a plurality of lancets and a plurality of test strips for the measurement of blood analytes;
a microcontroller operably coupled to the sampling and measurement device and capable of controlling the plurality of lancets and plurality of test strips relative to the measurement site; and
a set of electronic instructions executable by the microcontroller such that upon execution, the electronic instructions causes the microcontroller to initiate a sequence comprising selecting a lancet and test strip for use at the measurement site, firing the lancet to obtain a blood sample from the measurement site, collecting a blood sample from the measurement site; and depositing the blood sample onto the test strip;
wherein the microcontroller processes a electrochemical reaction from the test strip to determine the level of blood analytes and further wherein the electronic instructions causes the microcontroller to initiate the sequence without the need for manual intervention.

34. The automated system of claim 33, wherein the device comprises a sensor unit and a replaceable cartridge.

35. The automated system of claim 33, wherein the replaceable cartridge houses the consumable products.

36. The automated system of claim 33, wherein the electronic instructions for initiating the sequence are executed by the microcontroller upon command by an external controller.

37. The automated system of claim 33, wherein multiple blood sampling and measurement devices are linked to each other via a communications interface to obtain measurements from a plurality of measurement sites.

38. The automated system of claim 33, further comprising a monitoring device connected to a communications interface of the blood sampling and measurement device.

39. The automated system of claim 38, wherein the monitoring device controls initiation of measurements taken by the blood sampling and measurement device.

40. The automated system of claim 33, further comprising a treatment control device connected to a communications interface of the blood sampling and measurement device.

41. The automated system of claim 40, wherein the treatment control device controls initiation of measurements taken by the blood sampling and measurement device.

42. The automated system of claim 40, wherein the treatment control device automatically administers treatment to the patient based on the results obtained from the sampling and measuring device.

43. The automated system of claim 42, wherein the treatment to the patent includes automated infusion of one or more agents used to modify levels of blood analytes in the patient.

44. The automated system of claim 33, further comprising a monitoring and treatment system connected to the sampling and measuring device, wherein the monitoring and treatment system includes an intelligent control device and a multi-channel delivery device for providing an automated and controlled intravenous delivery of medications to affect the blood analyte levels in the patient.

45. A method for deploying an automated device for sampling and measuring blood analytes from a patient, comprising:
positioning an automated sampling and measuring device proximate to a measurement site on a patient, the sampling and measuring device configured to obtain blood analyte measurements from blood samples initiated with an automated process;
providing a set of replaceable materials to the automated sampling and measuring device, the set of replaceable materials including a plurality of reactive areas, each reactive area including one or more lancets and one or more test strips;
performing an automated blood analyte sampling and measurement using a blood sample obtained from the measurement site, the blood sample introduced to one of the plurality of the reactive areas provided to the automated sampling and measuring device; and
automatically repeating the step of performing a blood analyte measurement using a unused reactive area from the plurality of reactive areas, thereby performing a new blood analyte sampling and measurement at the measurement site without user intervention.

46. The method of claim 45, wherein during the automated blood analyte sampling and measurement the automated sampling and measuring device performs the steps of:
applying pressure proximate to the measurement site on the patient;
firing the one or more lancets to obtain the blood sample from the measurement site;
collecting the blood sample from the measurement site onto the one or more test strips;
retracting the one or more lancets from the measurement site;
electrochemically analyzing the one or more test strips; and
obtaining a blood analyte measurement from the electrochemical analysis of the one or more test strips.

47. The method of claim 46, further comprising executing a set of electronic instructions within the automated sampling and measuring device to perform the sampling and measurement steps.

48. The method of claim 46, further comprising automatically applying an anesthetic or analgesic solution to skin proximate to the measurement site prior to firing the one or more lancets.

49. The method of claim 45, further comprising verifying existence of an unused reactive area prior to performing a blood analyte sampling and measurement.

50. The method of claim 45, further comprising querying a status of the replaceable materials prior to performing a blood analyte sampling and measurement.

51. The method of claim 45, further comprising verifying placement of the automated sampling and measuring device at a valid measurement site prior to performing a blood analyte measurement.

52. The method of claim 45, wherein the blood analyte is selected from the group consisting of triglycerides, total cholesterol, HDL-cholesterol, fibrinogen, hemoglobin, ferritin, and glucose.

53. The method of claim 45, wherein the automated sampling and measuring device comprises a sensor unit including electronic circuitry and a microcontroller for conducting the automated performance of the blood analyte sampling and measurement.

54. The method of claim 53, wherein the microcontroller is connected via a communications interface to an external controller for initiating the automated performance of the blood analyte sampling and measurement.

55. The method of claim 53, wherein automatically repeating the step of performing a blood analyte measurement occurs by execution of a set of electronic instructions causing the microcontroller to initiate the blood analyte measurement iteratively over a period of time.

56. A method for sampling and measuring of blood analytes from a patient with an automated device, comprising:
affixing an automated sampling and measuring device to a patient, the sampling and measuring device accessing a supply of consumable products including a plurality of lancets and a plurality of test strips; and
executing a set of electronic instructions by a microcontroller within the sampling and measuring device, the execution of the electronic instructions causing the microcontroller to initiate a sequence for sampling and measuring a level of a blood analyte with the sampling and measuring device, the sequence including:
applying pressure proximate to a measurement site on the patient;
firing a lancet to obtain a blood sample from the measurement site;
exposing a test strip to the blood sample from the measurement site; and
obtaining an electrochemical measurement of the blood analyte level from the test strip;
wherein the set of electronic instructions for initiating the sampling and measuring the blood analyte level are executed by the microcontroller iteratively over a period of time or upon request, thereby enabling the sampling and measuring device to perform a series of automated sampling and measuring events without need for manual intervention.

57. The method of claim 56, wherein pressure proximate to the measurement site is removed after collecting a blood sample to prevent additional bleeding at the measurement site.

58. The method of claim 56, wherein pressure proximate to the measurement site is applied after collecting a blood sample to prevent additional bleeding at the measurement site.

59. The method of claim 56, wherein the sequence for sampling and measuring a level of a blood analyte further includes automatically applying an anesthetic or analgesic solution to skin proximate to the measurement site prior to firing the lancet.

60. The method of claim 56, wherein the sequence for sampling and measuring a level of a blood analyte further includes retracting the lancet from the measurement site.

61. The method of claim 56, further comprising verifying test supplies before executing the sequence.

62. The method of claim 61, wherein exhaustion of the consumable products is reported if test supplies are not verified.

63. The method of claim 56, wherein the request causing the initiation of a sequence for sampling and measuring is provided by a remote device connected via a communications interface to the microcontroller.

64. The method of claim 56, wherein the blood analyte is selected from the group consisting of triglycerides, total cholesterol, HDL-cholesterol, fibrinogen, hemoglobin, ferritin, and glucose.
